(19) United States
(12) Patent Application Publication
(10) Pub. No.: US 2009/0155232 A1
(43) Pub. Date: Jun. 18, 2009

(54) Neonatal human hepatocytes immortalized using tert and methods of their use

(75) Inventors: Jaya Gaddipati, Boyds, MD (US); Judith Kantor, Rockville, MD (US); Yvonne A. Reid, Haymarket, VA (US)

Correspondence Address: LAHIVE & COCKFIELD, LLP;FLOOR 30, SUITE 3000, ONE POST OFFICE SQUARE, BOSTON, MA, 02109, US

(73) Assignee: American Type Culture Collection, Manassas, VA (US)

(21) Appl. No.: 12/218,301

(22) Filed: Jul. 14, 2008

Related U.S. Application Data
(63) Continuation of application No. 11/901,509, filed on Sep. 18, 2007, now abandoned.
(60) Provisional application No. 60/845,618, filed on Sep. 19, 2006.

(51) Int. Cl. A61K 48/00; C12N 5/10; C12N 15/85; C12Q 1/02

(52) U.S. Cl. 424/93.21; 435/370; 435/455; 435/29

(57) ABSTRACT

The present invention relates to the discovery of immortalized neonatal human hepatocytes that exhibit phenotypic features of human hepatic progenitor cells. The invention is also directed to a method of obtaining telomerase-immortalized neonatal human hepatocytes that exhibit phenotypic features of human hepatic progenitor cells. Furthermore, the instant invention describes methods of using the immortalized neonatal human hepatocytes in cellular therapies, toxicological studies, pharmacokinetic studies, metabolic studies, therapeutic gene delivery and for the production of fully differentiated hepatocytes.

### RELATED APPLICATION

This application also claims priority to U.S. application Ser. No. 11/901,509 filed Sep. 18, 2007, the entire contents of which are expressly incorporated herein by reference. This application also claims priority to U.S. provisional application Ser. No. 60/845,618 filed Sep. 19, 2006, the contents of which are entirely incorporated by reference.

### BACKGROUND OF THE INVENTION

Liver-directed cell therapies, including hepatocyte transplantation and bioartificial liver support, constitute promising alternatives to whole-liver transplantation. However, current techniques to expand human hepatic cells in vitro are inadequate. There is a lack of cell lines that display the full spectrum of hepatic progenitor phenotypic features and existing cell lines are often only stable for a limited number of population doublings. In an effort to overcome the restricted in vitro proliferative capacity of these cells, different immortalization techniques have been investigated, including the introduction of the Simian virus 40 large T-antigen (Kobayashi et al., (2000) Science 287:1258-62, Nakamura et al., (1997) Transplantation 63 (11):1541-47), transfection of antisense constructions against p53 and retinoblastoma protein (Werner et al., (2000) Biotechnol Bioeng 68 (1): 59-70), transgenic introduction of a truncated Met protein (Amicone et al., (1997) EMBO J. 16 (3):495-503), and expression of a hepatitis C virus core protein (Ray et al., (2000) Virology 271:197-204).

The mechanism restricting the in vitro proliferation of human fibroblast cells has been shown to be the progressive shortening of telomeres with each cell division (Hayflick et al., (1961) Exp. Cell Res. 25:585-621). Telomeres constitute the terminal regions of chromosomes, and shortened telomeres trigger the restriction of proliferation. Stem cells, however, are able to avoid telomere-dependent proliferative restriction by adding telomeric repeat sequences onto chromosome ends using telomerase reverse transcriptase (Greider et al., (1985) Cell 43:405-413). The ability to achieve telomerase reconstitution in hepatocytes to develop stable human hepatocyte-derived cell lines having the phenotypic characteristics of hepatic progenitor cells after passage in vitro for use, e.g., in liver-directed cell therapies and toxicological studies, would be of great benefit.

### SUMMARY OF THE INVENTION

The present invention relates to immortalized neonatal human hepatocytes. These immortalized cells are obtained by the reconstitution of human telomerase (hTERT) in neonatal human hepatocyte cells. Ectopic expression of human telomerase reverse transcriptase is one of the major strategies used in developing immortalized cells. It allows for the retention of original cellular characteristics, and avoids some of the problems associated with other approaches. Transfection can be performed using, for example, a retroviral vector system.

The instant invention is based, at least in part, on the surprising discovery that these immortalized neonatal human hepatocytes exhibit the phenotypic features of hepatic progenitor cells. This is true both in early and late passage in vitro. Furthermore, such immortalized cells maintain a diploid karyotype and expressed gene product profiles similar to normal neonatal human hepatocytes. These features are desirable when developing human hepatocyte-derived cell lines for use, e.g., in cellular therapies, toxicological studies, pharmacokinetic studies, metabolic studies, and therapeutic gene delivery. Furthermore, these hepatic progenitor cells are useful for the production of fully differentiated hepatocytes or bile duct cells. Thus, they provide a readily available source of differentiated hepatocytes or biliary cells that can be used in a variety of applications.

In one aspect, the invention provides a population of immortalized human cells that express a human telomerase, wherein the population exhibits phenotypic features of human hepatic progenitor cells at early passage and continues to express said phenotypic features at late passage in vitro. In another aspect, the invention provides an immortalized human cell that expresses a human telomerase, wherein the cell exhibits phenotypic features of human hepatic progenitor cells at early passage in vitro and continues to express said phenotypic features at late passage in vitro. In one embodiment, the immortalized cell may be used to produce fully differentiated hepatocytes. In one embodiment, the immortalized cell expresses Cytokeratin 19. In another embodiment, the immortalized cell expresses Cytokeratin 19 at a high level. In another embodiment, the immortalized cell expresses Albumin at a low level. In another embodiment, the cell expresses c-kit. In yet another embodiment, the cell expresses Cytokeratin 19 and expresses Albumin at a low level. In another embodiment, the cell expresses Cytokeratin 19, expresses Albumin at a low level and expresses c-kit. In yet another embodiment, the phenotype of the cell is characterized by one or more of: expression of Cytokeratin 19, expression of Neuronal cell adhesion molecule (NCAM), expression of Epithelial cell adhesion molecule (EpCAM), expression of Claudin-3 (CLDN-3); low expression of Albumin; the absence of expression of alpha-fetoprotein, the absence of expression of Asialoglycoprotein receptor (ASGP-R), and the absence of expression of Cytochrome P450 3A4 (CYP 3A4).

In another embodiment, the telomerase is encoded by a human TERT (hTERT) gene. In yet another embodiment, the cell is diploid. In one embodiment, the cell is transfected with a retroviral gene transfer and expression system containing the hTERT gene. In another embodiment, the retroviral gene transfer and expression system is pBABE Puro. In yet another embodiment, the retroviral gene transfer and expression system is pLXSN. In one embodiment, the retroviral gene transfer and expression system is pMSCV.

In another aspect, the invention provides a method of obtaining an immortalized human cell or population of cells that express a human telomerase, wherein the cell or population of cells exhibit phenotypic features of human hepatic progenitor cells, wherein said phenotypic features include the expression of Cytokeratin 19. In one embodiment, the cell or population of cells exhibit phenotypic features in early passage in vitro. In another embodiment, the cell or population of cells exhibit phenotypic features in middle passage in vitro. In yet another embodiment, the cell or population of cells exhibit phenotypic features in late passage in vitro. In another embodiment, the immortalized cell expresses Cytokeratin 19 at a high level. In another embodiment, the immortalized cell expresses Albumin at a low level. In another embodiment, the cell expresses c-kit. In yet another embodiment, the cell expresses Cytokeratin 19 and expresses Albumin at a low level. In another embodiment, the cell expresses Cytokeratin 19, expresses Albumin at a low level and expresses c-kit. In yet another embodiment, the phenotype of the cell is characterized by one or more of: expression of Cytokeratin 19, expression of NCAM, expression of EpCAM, expression of CLDN-3; low expression of Albumin; the absence of expression of alpha-fetoprotein, the absence of expression of ASGP-R, and the absence of expression of CYP 3A4.

In another aspect, the invention provides a method of obtaining an immortalized human hepatocyte having the phenotypic features of human hepatic progenitor cells, said method comprising the steps of: introducing an exogenous nucleic acid molecule encoding a human telomerase into a neonatal human hepatocyte to obtain a transfected neonatal human hepatocyte cell that expresses the endogenous human telomerase; and propagating the transfected human hepatocyte cell in vitro, to thereby obtain an immortalized neonatal human hepatocyte cell that exhibits phenotypic features of human hepatic progenitor cells at early passage in vitro and continues to express said phenotypic features at late passage in vitro. In one embodiment, the nucleic acid molecule comprises a retroviral gene transfer and expression system and a telomerase. In another embodiment, the telomerase is encoded by a human TERT gene. In yet another embodiment, the retroviral gene transfer and expression system is pBABE Puro. In yet another embodiment, the retroviral gene transfer and expression system is pLXSN. In yet another embodiment, the retroviral gene transfer and expression system is pMSCV.

In one embodiment, the immortalized cell expresses Cytokeratin 19. In another embodiment, the immortalized cell expresses Cytokeratin 19 at a high level. In another embodiment, the immortalized cell expresses Albumin at a low level. In another embodiment, the cell expresses c-kit. In yet another embodiment, the cell expresses Cytokeratin 19 and expresses Albumin at a low level. In another embodiment, the cell expresses Cytokeratin 19, expresses Albumin at a low level and expresses c-kit. In yet another embodiment, the phenotype of the cell is characterized by one or more of: expression of Cytokeratin 19, expression of NCAM, expression of EpCAM, expression of CLDN-3; low expression of Albumin; the absence of expression of alpha-fetoprotein, the absence of expression of ASGP-R, and the absence of expression of CYP 3A4.

In yet another aspect, the invention provides a method of ameliorating at least one symptom of disease in an individual in need thereof, said method comprising the step of: transplanting to an individual an immortalized neonatal human hepatocyte, whereby at least one symptom of hepatic disease is ameliorated.

In another aspect, the invention provides a method of evaluating the toxicity of a compound, said method comprising the steps of: contacting an immortalized neonatal human hepatocyte with a compound; measuring the toxicity of the compound for the immortalized cells; to thereby evaluate the toxicity of a compound.

In yet another aspect, the invention provides a method of evaluating the pharmacokinetics of a compound in vitro, said method comprising the steps of: contacting the immortalized cell with a compound; measuring the pharmacokinetics of the compound for the immortalized cells; to thereby evaluate the pharmacokinetics of a compound.

In yet another aspect, the instant invention provides a method of evaluating the metabolism of a compound, said method comprising the steps of: contacting the immortalized cell with a compound; measuring the metabolasis of the compound for the immortalized cells; to thereby evaluate the metabolism of a compound.

In another aspect, the invention provides a method of delivering a therapeutic gene to a patient having a condition amenable to gene therapy comprising selecting the patient in need thereof; introducing a therapeutic gene into the immortalized cell of claims 1 or 2 to obtain a modified cell or population of cells; and administering the modified cell or population of cells to the patient.

In yet another aspect, the invention provides a commercial package comprising the immortalized cell or population of cells of claims 1 or 2, wherein a therapeutic gene has been introduced into the immortalized cell or population of cells to obtain a modified cell or population of cells, and instructions for treating a patient having a condition amendable to treatment with gene therapy.

### BRIEF DESCRIPTION OF THE DRAWINGS

FIG. 1: Morphology of the hTERT-immortalized neonatal human hepatocytes, at early passage (passage 8) and late passage (passage 25-26).

FIG. 2: Assay for telomerase activity of the hTERT-immortalized neonatal human hepatocytes at early passage by TRAP assay.

FIG. 3: Assay for telomerase activity of the hTERT-immortalized neonatal human hepatocytes at late passage by TRAP assay.

FIG. 4: Gene expression analysis by RT-PCR of the hTERT-immortalized neonatal human hepatocytes at early and late passage.

FIG. 5: Gene expression analysis by RT-PCR of the hTERT-immortalized neonatal human hepatocytes at early and late passage.

Other features and advantages of the invention will be apparent from the following detailed description and claims.

### DETAILED DESCRIPTION OF THE INVENTION

The instant invention is based, at least in part, on the surprising discovery that immortalized neonatal human hepatocytes exhibit phenotypic features specific to human hepatic progenitor cells even after prolonged passage in vitro. These immortalized cells are obtained by the reconstitution of human telomerase (hTERT) in neonatal human hepatocyte cells. Ectopic expression of human telomerase reverse transcriptase is one of the major strategies used in developing immortalized cells; it allows for the retention of original cellular characteristics, and avoids some of the problems associated with other approaches. Transfection can be performed using, for example, a retroviral vector system.

The advantages of such immortalized neonatal human hepatocytes include the fact that they can provide cells with the phenotypic functions of hepatic progenitor cells. Furthermore, such immortalized cell lines maintain a diploid karyotype and expressed gene product profiles similar to normal neonatal human hepatocytes. Such immortalized cell lines preserve the normal biological characteristics of neonatal hepatocytes and may therefore be useful, among other things, in liver-directed cell therapies, toxicological studies, pharmacokinetic studies, metabolism studies, and therapeutic gene delivery.

So that the invention may be more readily understood, certain terms are first defined.

The term “hepatocyte,” as used herein, means a predominant cell of the liver responsible for the synthesis, degradation and storage of a wide range of substances within the liver. Hepatocytes are the site of synthesis of plasma proteins, other than antibodies, and are the site of storage of glycogen. Within the liver, but not necessarily when propagated in cell culture, hepatocytes are arranged in folded sheets facing blood-filled spaces called sinusoids.

The term “hepatoblast,” as used herein, is defined as the precursor for hepatocytes as well as for cholangiocytes, the cells that form the biliary ductal system of the liver. Hepatoblasts express Albumin and alpha-fetoprotein, have low expression of CK19 and ASGP-R, and do not express N-CAM or CLDN-3.

The term “immortalized cell” or “immortal cell” refers to any cells that are not limited by the Hayflick limit.

The term “Hayflick limit,” as used herein, is defined as the number of times that differentiated cells can divide (e.g., about 50 times) before dying. As cells approach this limit, they show signs of aging. The number of times a cell divides varies from cell type to cell type, however, the human cell limit is around 52. The Hayflick limit has been linked to the shortening of telomeres and is believed to be one of the causes of cellular aging and senescence. It is believed that if the shortening of telomeres can be slowed or prevented, life expectancy can be extended.

The term “differentiated cell” or “differentiated” refers to a cell that has developed specific structures and perform specific functions. A differentiated cell is specialized and cannot develop into any other type of cell. Differentiated cells are characterized by numerous aspects of cell physiology, including size, shape, polarity, metabolic activity, responsiveness to signals, and gene expression profiles.

“Differentiation” in the present context means the formation of cells expressing markers known to be associated with cells that are more specialized and closer to becoming terminally differentiated cells incapable of further division or differentiation.

The term “proliferation” indicates an increase in cell number.

The term “progenitor cell” refers to cells that are either pluripotent, bipotent, or multipotent and capable of multiple rounds of replication. A progenitor cell is a parent cell that can give rise to a distinct cell lineage by a series of cell divisions. The term “progenitor cell” can be used synonymously with “stem cell.” Both terms refer to a cell that has not completely differentiated, which is capable of proliferation and giving rise to more progenitor cells having the ability to generate a large number of mother cells that can, in turn, give rise to differentiated, or differentiable daughter cells. In a preferred embodiment, the term progenitor or stem cell refers to a generalized parent cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors.

The term “stem cell” as used herein refers to a cell typically characterized by its capacity for self-renewal and ability to give rise to multiple differentiated cellular populations. A stem cell is not limited by the Hayflick limit. Pluripotential stem cells, adult stem cells, blastocyst-derived stem cells, gonadal ridge-derived stem cells, teratoma-derived stem cells, totipotent stem cells, multipotent stem cells, embryonic stem cells (ES), embryonic germ cells (EG), and embryonic carcinoma cells (EC) are all examples of stem cells. In one embodiment, a stem cell or progenitor cell or a population of such cells may be derived from the liver. In one embodiment such a stem or progenitor cell or population may be pluripotent. In another embodiment, such a stem or progenitor cell or population may be a hepatic progenitor or stem cell and may be able to differentiate, e.g., into a mature hepatocyte(s). In one embodiment, such differentiation may be accomplished by altering the growth condition of the cell, e.g., by contacting the cells with one or more factors that induce or promote differentiation.

The term “cell” as used herein refers to individual cells, populations of cells, cell lines, primary culture, or cultures derived from such cells unless specifically indicated otherwise. A “culture” refers to a composition comprising isolated cells of the same or a different type. A cell line is a culture of a particular type of cell that can be reproduced indefinitely, thus making the cell line “immortal.”

The term “telomerase,” as used herein, is defined as an enzyme that adds specific DNA sequence repeats to the 3′ end of DNA strands in the telomere region at the end of chromosomes. The telomerase enzyme is a reverse transcriptase that carries its own RNA template for DNA replication. Human telomerase is composed of two subunits, human Telomerase Reverse Transcriptase (hTERT or TERT) and human Telomerase RNA (hTR). These two subunits are encoded by two genes. hTERT mRNA is identified in Genebank as either Accession No. NM_{—}198253 or NM_{—}198255.

The term “hepatic progenitor cell” refers to a cell which can differentiate into a cell of hepatic lineage, e.g., a cell which can produce a hormone or enzyme normally produced by a hepatic cell. For instance, a pancreatic progenitor cell may be caused to differentiate, at least partially, into hepatoblasts, bone marrow cells, oval cells, hepatocytes, hepato-pancreatic stem cells, and/or mature hepatocytes. The hepatic progenitor cells of the invention can also be cultured prior to administration to a subject under conditions which promote cell proliferation and differentiation.

The term “phenotypic features of human hepatic progenitor cells” refers to the distinct, observable characteristics of a cell as distinct from its genotype. The phenotypic features of human hepatic progenitor cells refer to distinct, observable characteristics, including the expression of CK19, NCAM, EpCAM, CLDN-3, and c-kit, the low expression of Albumin, and the absence of expression of AFP and adult liver specific proteins (Table 1).

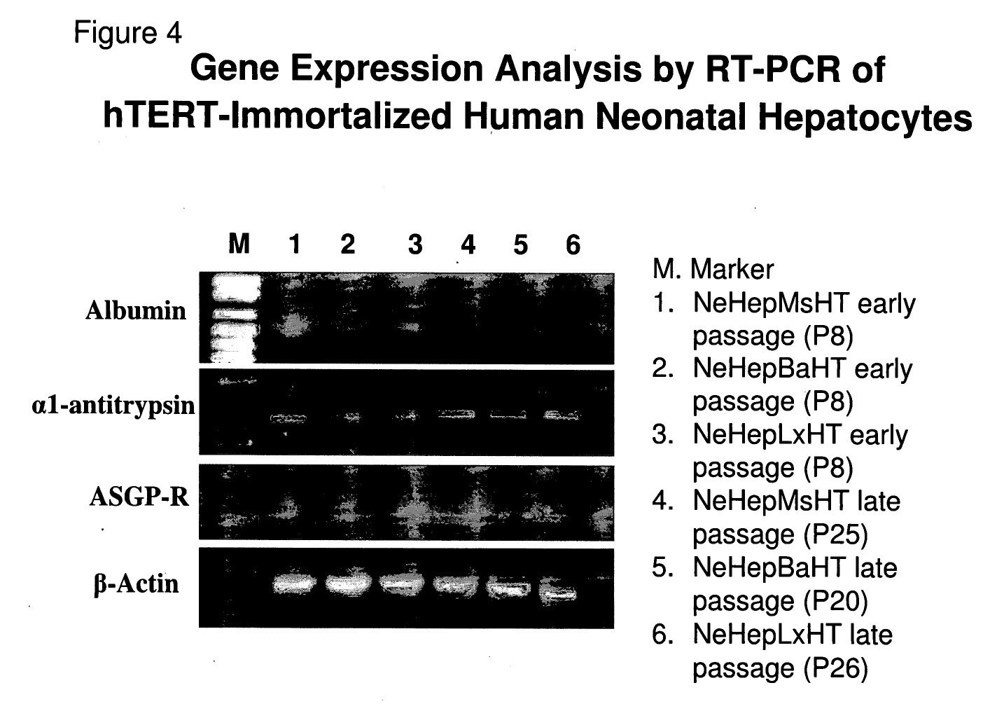

The term “low” or “reduced” means downmodulating an event or characteristic. It is understood that this is typically in relation to some standard or expected value, in other words it is relative, but that it is not always necessary for the standard or relative value to be referred to. For example, “low expression” means lowering the amount of gene expression that takes place relative to a standard or a control. In one embodiment, gene expression may be low or reduced relative to a standard or a control. Exemplary controls used herein include GAPDH and β-actin. For example, the expression of Albumin was observed to be “low” as compared to the housekeeping gene β-actin in FIG. 4. In another embodiment, examples of a standard or a control can include an alternate cell line, e.g., HepG2. For example, the expression of Albumin in the immortalized neonatal human hepatocytes has been observed to be “low” as compared to the expression of Albumin in the HepG2 cell line.

The term “high” or “increased” refers to upmodulating an event or characteristic. It is understood that this is typically in relation to some standard or expected value, in other words it is relative, but that it is not always necessary for the standard or relative value to be referred to. For example, “high expression” means increasing the amount of gene expression that takes place relative to a standard or a control. In one embodiment, gene expression may be high or increased relative to a standard or a control. Exemplary controls used herein include GAPDH and β-actin. For example, the expression of CK19 was observed to be “high” as compared to the housekeeping gene GAPDH in Table 2. In another embodiment, examples of a standard or a control can include an alternate cell line, e.g., HepG2. For example, the expression of CK19 in the immortalized neonatal human hepatocytes can be compared to the expression of CK19 in the HepG2 cell line.

The term “early passage” refers to cells that have been passaged at least about (or about) 1, 2, 3, 4, 5, 6, 7, 8, 9, 10, 11, 12, 13, or 14 passages. In various embodiments, early passage ranges between about 8 passages to about 10 passages.

The term “late passage” refers to cells that have been passaged at least about (or about) 18, 19, 20, 21, 22, 23, 24, 25, 26, 28, 28, 29, or 30 passages. In one embodiment, late passage refers to cells that have been passaged at least about 18 times. In various embodiments, late passage ranges between about 20 passages to about 26 passages. In other embodiments, late passage can include 100 passages or more.

The term “middle passage” refers to cells that have been passaged at least about (or about) 15, 16, or 17 passages. In one embodiment, middle passage refers to cells that have been passaged at least about 16 times.

As used herein, the term “treatment” or “treating” is defined as the application or administration of a therapeutic agent, e.g., a cell or population of cells, to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

The term “alpha-fetoprotein” or “AFP” refers to both glycosylated and non-glycosylated proteins from the serum of vertebrate embryos, which likely serve as albumins. For example, human alpha-fetoprotein includes secreted forms of the human alpha-fetoprotein precursor. An exemplary sequence of AFP mRNA is found in Genebank as Accession No. NM_{—}001134, and an exemplary sequence of AFP protein is found in Genebank as Accession No. NP_{—}001125.1.

The term “Cytokeratin 19” or “CK19” refers to a single, non-glycosylated polypeptide chain having a molecular mass of 44 kDa. CK19 is not expressed in most differentiated hepatocytes, and is therefore useful in the classification of hepatic stem cells or the identification of liver metastases. CK19 is expressed at a high level in hepatic stem cells, a low level in hepatoblasts, and is not expressed in mature hepatocytes. An exemplary sequence of CK19 mRNA is found in Genebank as Accession No. NM_{—}002276, GI 40217850, and an exemplary sequence of CK19 protein is found in Genebank as Accession No. NP_{—}002267, GI No. 24234699.

The term “Albumin” refers to a protein which is the most abundant plasma protein and is important for transporting fatty acids, thyroid hormones, and other substances. Albumin is expressed at a high level in hepatoblasts and mature hepatocytes and at a low level in hepatic stem cells. Exemplary sequences of Albumin are well known.

The term “c-kit” refers to a member of the PDGFR family. C-kit is a tyrosine kinase receptor that dimerizes following ligand binding and is autophosphorylated on intracellular tyrosine residues. C-kit is expressed in hepatic stem cells. Exemplary sequences of c-kit are well known in the art.

The term “NCAM” or “Neuronal cell adhesion molecule” is defined as a homophilic binding glycoprotein that is expressed on the surface of hepatic stem cells, neurons, glia, and skeletal muscle. It has been shown that NCAM may play a role in cell to cell adhesion, neurite outgrowth, synaptic plasticity, learning, and memory. NCAM is highly expressed in hepatic stem cells but is not expressed in hepatoblasts or mature hepatocytes. An exemplary sequence of NCAM protein is found in Genebank as Accession No. NP_{—}000606, and an exemplary sequence of NCAM mRNA is found as Genebank Accession No. NM_{—}000615.

As used herein, the term “EpCAM” or “Epithelial cell adhesion molecule” refers to a 40 kDa type I transmembrane glycoprotein that consists of two epidermal growth factor-like extracellular domains, a cysteine-poor region, a transmembrane domain, and a short cytoplasmic tail. It is encoded by the GA733-2 gene on the long arm of chromosome 4 and is involved in cell to cell adhesion. EpCAM is expressed on the majority of epithelial tissues, with the exception some epithelium-derived cells, including hepatocytes. EpCAM is highly expressed in hepatic stem cells. Exemplary sequences of EpCAM are well known in the art.

The term, “CLDN-3” or “Claudin-3,” as used herein, refers to a protein essential for the formation of tight junctions in epithelial and endothelial cells. Claudin-3 is highly expressed in hepatic stem cells but is not expressed in hepatoblasts or mature hepatocytes. An exemplary sequence of CLDN-3 protein is found in Genebank Accession No. NP_{—}001297, and an exemplary sequence of CLDN-3 mRNA is found in Genebank Accession No. NM_{—}001306.

As used herein, the term “ASGP-R” refers to a 42 kDa transmembrane glycoprotein that mediates binding, internalization, and degradation of extracellular glycoproteins with exposed terminal galactose residues. ASGP-R is expressed on the surface of hepatocytes in a polar manner, i.e., it is present on the sinusoidal and lateral plasma membranes, but not on the bile canalicular membrane. The mammalian hepatic ASGP-R mediates the endocytosis and degradation of serum proteins from which terminal sialic residues have been removed. ASGP-R is not expressed on hepatic stem cells, is expressed at a low level of hepatoblasts, and is expressed at a high level on mature hepatocytes. An exemplary sequence of ASGP-R protein is found in Genebank Accession No. NP_{—}001662, and an exemplary sequence of ASGP-R mRNA is found in Genebank Accession No. NM_{—}001671.

The term “CYP 3A4” is defined as an enzyme involved in the metabolism of xenobiotics in the body, the oxidation of a range of substrates of all of the cytochromes, and present in a large quantity of all the cytochromes in the liver. CYP 3A4 is not expressed in hepatic stem cells. An exemplary sequence of CYP 3A4 protein is found in Genebank Accession No. NP_{—}059488, and an exemplary sequence of CYP 3A4 mRNA is found in Genebank Accession No. NM_{—}017460.

The term “diploid” or “diploid cell” refers to a cell that has two sets of chromosomes, one from each parent.

As used herein, the term “retroviral gene transfer and expression system” is defined as a retroviral system that transmits a cloned gene of interest into a target cell. Once in the cell, RNA from the vector is packaged into infectious, replication-incompetent retroviral particles. The retroviral gene transfer and expression system then transmits the gene of interest, which is cloned between the viral LTR sequences, into the chromosome of the target cell. The retrovirus cannot replicate within the target cell, however, since it lacks viral structural genes. The retroviral gene transfer and expression system of the instant invention can include any known retroviral vector. In a preferred embodiment, the retroviral vector is pBABE puro, pLXSN, or pMSCV.

The term “pBABE puro” refers to a retroviral gene transfer and expression system that is based on the Moloney Murine Leukemia Virus. pBABE puro is typically utilized to transfer genetic material to the broadest possible range of mammalian cells and has been shown to have stable expression in mammalian cells.

The term “pLXSN,” as used herein, refers to a retroviral gene transfer and expression system that contains elements derived from Moloney murine leukemia virus and Moloney murine sarcoma virus. pLXSN is designed for retroviral gene delivery and expression. Upon transfection into a packaging cell line, pLXSN can transiently express, or integrate and stably express, a transcript containing the gene of interest and a selectable marker. pLXSN is typically used to efficiently transfer genetic material for stable expression in a broad range of mammalian cells, has been shown to transduce nearly 100% of cells with retrovirus-mediated gene transfer, and easily creates stable cell lines.

As used herein, the term “pMSCV” refers to a retroviral gene transfer and expression system that is derived from Murine Embryonic Stem Cell Virus and the LN retroviral vectors. Upon transfection into a packaging cell line, pMSCV can transiently express, or integrate and stably express, a transcript containing the gene of interest and a selectable marker. pMSCV is typically used to transfer genetic material to pluripotent (ES) cell lines and has been optimized for stable expression in human and mouse hematopoietic, embryonic stem, and embryonal carcinoma cells.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Effective amounts can readily be determined by one of skill in the art.

The term “patient” includes human and other mammalian subjects that receive either prophylactic or therapeutic treatment.

As used herein, the term “isolated” molecule (e.g., isolated nucleic acid molecule) refers to molecules which are substantially free of other cellular material, or culture medium when produced by recombinant techniques, or substantially free of chemical precursors or other chemicals when chemically synthesized.

Various aspects of the invention are described in further detail in the following subsections.

### Hepatic Cell Immortalization

According to the invention, immortalized cells are obtained by the reconstitution of telomerase in the cells, e.g., neonatal mammalian hepatocytes. In one embodiment, the neonatal hepatocytes are human.

The cells for immortalization are derived from neonatal livers. For example, the cells may be isolated using standard procedures and either cryopreserved or maintained in primary culture until use. Primary neonatal hepatocytes can be harvested from neonatal liver donors. Exemplary procedures for isolating and culturing primary human hepatocytes from human livers are described in Strom, et al. (1996) Methods Enzymol 272:388-401.

Isolated neonatal cells from a tissue or organ of interest, maintained in culture, are provided with nucleic acid encoding a telomerase catalytic subunit of human telomerase reverse transcriptase (hTERT). The human telomerase catalytic subunit has been cloned previously (see Nakamura, et al. (1997) Science 277: 955; Mayerson, et al. (1997) Cell 90: 78; and Kilian, et al. (1997) Hum Mol Genet. 6: 2011; U.S. Pat. No. 6,166,178). Sources of the coding sequence for the human telomerase subunit include any cells that demonstrate telomerase activity such as immortal cell lines, tumor tissues, germ cells, proliferating stem or progenitor cells, and activated lymphocytes. The nucleic acid can be obtained using methods known in the art.

The manner in which the hTERT coding region is introduced into the cells of interest is not critical, as long as a functional telomerase catalytic subunit is expressed. Expression can be extrachromosomal or following integration into the cellular genome. Any of a variety of techniques can be used to introduce the hTERT gene into the desired cells, including electroporation, liposomes, or viral vectors. See Molecular Cloning, 3rd Edition, 2001, by Sambrook and Russell.

A preferred means for incorporating the hTERT coding region into the cells of interest is to use a recombinant retrovirus that provides for integration of the hTERT efficiently and stably into the genome of the target cell. Since one intended use of the immortalized cells is in human therapy, it is important that the retrovirus used is replication-defective and not contaminated with wild-type viruses (Temin, (1990) Hum. Gene Ther. 1: 111).

The recombinant retroviruses which are used are derived from viruses with a natural host-specificity that includes primates, or from viruses that can be pseudotyped with a host-specificity that includes primates. Such viruses include, murine leukemia viruses (MuLV; Weiss et al., (1984) RNA Tumor Viruses, New York) with a so-called amphotropic or xenotropic host-range, gibbon ape leukemia viruses (GaLV; Lieber et al., Proc. Natl. Acad. Sci. USA 72 (1975) 2315-2319), and primate lentiviruses. For the production of recombinant retroviruses, two elements are required: the so-called retroviral vector, which, in addition to the gene (or genes) to be introduced, contains all DNA elements of a retrovirus that are necessary for packaging the viral genome and the integration into the host genome; and the so-called packaging cell line which produces the viral proteins that are necessary for building up an infectious recombinant retrovirus (Miller, (1990), Hum. Gene Ther. 1: 5).

The packaging cell lines used should be those constructed so that the risk of recombination events whereby a replication-competent virus is generated, are minimized. This generally is effected by physically separating into two parts the parts of the virus genome that code for viral proteins and introducing them into the cell line separately (Danos and Mulligan, (1988) Proc. Natl. Acad. Sci. USA 85: 6460; Markowitz et al., (1988) J. Virol. 62: 1120; and Markowitz et al., (1988) Virology 167: 400). As the presence of both constructs is essential to the functioning of the packaging cell line and chromosomal instability occurs regularly, it is important for use of such cells in procedures related to human therapy that, by means of a selection medium, selection for the presence of the constructs can be provided for. Therefore, these constructs generally are introduced by means of a cotransfection whereby both viral constructs are transfected together with a dominant selection marker.

The retroviral vectors generally include retroviral long terminal repeats, packaging sequences, and cloning site(s) for insertion of heterologous sequences as operatively linked components. Other operatively linked components may include a nonretroviral promoter/enhancer and a selectable marker gene. Examples of retrovirus expression vectors which can be used include pLXSN, pBabe-puro (Proc. Natl. Acad. Sci. (1995) 92: 9146-9150) and pMSCV. The retrovirus expression vector, pBabe-puro-hTERT, is also available and already includes the hTERT gene (Morgenstern and Land, 1990). In some instances, it may be desirable to increase expression of the hTERT gene by utilizing other promoters and/or enhancers in place of the promoter and/or enhancers provided in the expression vector. These promoters in combination with enhancers can be constitutive or regulatable. Any promoter/enhancer system functional in the target cell can be used.

To package the recombinant retrovirus vectors containing the nucleic acid-to-be-expressed, cells lines are used that provide in trans the gene functions deleted from the recombinant retrovirus vector such that the vector is replicated and packaged into virus particles. The genes expressed in trans encode viral structural proteins and enzymes for packaging the vector and carrying out essential functions required for the vector's expression following infection of the target host cell. Packaging cell lines and retrovirus vector combinations that minimize homologous recombination between the vector and the genes expressed in trans are preferred to avoid the generation of replication competent retrovirus. Packaging systems that provide essential gene functions in trans from co-transfected expression vectors can be used and packaging systems that produce replication competent retroviruses. Following packaging, the recombinant retrovirus is used to infect target cells of interest. The envelope proteins expressed should permit infection of the target cell by the recombinant retrovirus particle. Retrovirus packaging cell lines which can be used include BOSC23 (Proc. Natl. Acad. Sci. (USA) 90: 8392-8396), PT67 (Miller and Miller. 1994. J. Virol. 68: 8270-8276, Miller. 1996. Proc. Natl. Acad. Sci. (USA) 93: 11407-11413), PA317. (Mol. Cell. Biol. 6: 2895 (1986)), PG13, 293 cells transfected with pIK6.1 packaging plasmids (U.S. Pat. No. 5,686,279), GP+envAM12 (Virology 167: 400 (1988), PE502 cells (BioTechniques 7: 980-990 (1989)), GP+86 (Markowitz, et al. 1988. J. Virol. 62: 1120-1124), PSI-Cre (Danos and Mulligan. 1988. Proc. Natl. Acad. Sci. (USA) 85: 6460-6464). The preferred titer of recombinant retrovirus particles is about 10⁵-10⁷ infectious particles per milliliter. If these titers cannot be achieved the virus also can be concentrated before use.

For transfection, the neonatal human hepatocytes or other cells to be transfected may be suspended in a suitable culture medium containing recombinant retrovirus vector particles. Many different suitable culture media are commercially available. They include DMEM, IMDM, and α-MEM, with 5-30% serum and often further supplemented with, e.g., BSA, one or more antibiotics and optionally growth factors suitable for stimulating cell division. Recombinant retrovirus vector particles are harvested into this medium by incubating the virus-producing cells in this medium. To enhance gene transfer, compounds such as polybrene, protamine sulphate, or protamine HCl generally are added. Usually, the cultures are maintained for 2-4 days and the recombinant retrovirus vector containing medium is refreshed daily. Optionally, the cells to be transfected are precultured in medium with growth factors but without recombinant retrovirus vector particles for up to 2 days, before adding the recombinant retrovirus vector containing medium. For successful gene transfer it is essential that the target cells undergo replication in culture (without differentiation).

Successfully transduced cells may be selected by culturing cells in medium containing a selection drug (e.g., puromycin or G418) that allows permissive growth only by cells that express an appropriate selection marker gene, and are analyzed for mRNA levels of the telomerase catalytic unit, using RT-PCR, particularly real-time RT-PCR oftentimes used in evaluating telomerase activity, or by using commercially available kits (Roche Molecular Biochemicals) or other techniques known in the art.

Telomerase activity of transfected cells may be determined using any of the myriad variations of telomeric repeat amplification protocol (TRAP) assays in the literature and known to those in the art. A non-amplified or a PCR-based assay can be applied (Kim and Wu 1997). TRAP assays that utilize radiochemical—(i.e., ³²P) or enzyme-(ELISA) based detection can be applied. Telomere length comparisons between transduced and non-transduced cells are carried out by isolating genomic DNA and then digesting with a restriction enzyme that does not cut within the telomeres (for example, HinfI and RsaI). The undigested telomeres are then labeled (with a radiochemical, a fluorescent compound, or an enzyme) and resolved in a gel. In applications where it is desired, once it is established that the hTERT coding sequence is incorporated into the genomic DNA, it is preferred to maintain the cells in the absence of selective drug. The selective drug is removed before the cells are used therapeutically.

To confirm that the transduced cells have been immortalized, the phenotype of hTERT-transduced cells is evaluated and compared to non-transduced cells, and transformed immortal cells. Transduced cells are less susceptible to induction of apoptosis and do not develop staining characteristics associated with senescence, retaining normal chromosome patterns. Telomerase immortalized cells do not acquire morphologic or phenotypic changes generally associated with cancer cells, yet their growth curves are similar to those of transformed cell lines.

Proliferative capacity of the transduced cells may be compared to untransduced control cells. For example, the cells are grown in monolayers until cell cycle arrest or immortality can be confirmed (2-fold increase in doubling potential). The number of population doublings (PD) is estimated by the count/split-method (Vaziri and Benchimol S (1998) Curr Biol 8: 279-282). Growth curves are generated for the cultured cells and time to confluency is determined. β-galactosidase can serve as a biomarker to visualize senescence in hepatocyte cultures (Dimri et al., (1995) Proc Natl Acad Sci USA 92: 9363-9367). 3H-thymidine incorporation (18) and a BrdU incorporation-based flow cytometry assay (BrdU Flow Kit, PharMingen) are used to detect DNA synthesis and to characterize the cell cycle distribution of the cultured cells.

### Characteristics of Immortalized Hepatic Cells

Cells immortalized using these methods have the characteristics of progenitor or stem cells. The advantages of these telomerase-immortalized neonatal human hepatocytes include that they can provide all of the functions of a progenitor cell, without the undesirable risks associated with oncogene-immortalized cells or xenogenic cells. They do not develop a transformed or differentiated phenotype, even after extended population doublings (e.g., during late passage). Using telomerase-immortalized cells, such as neonatal hepatocytes, for direct transplantation procedures has the benefit of greatly reduced cost, of providing endogenous organ function to a much greater number of individuals and of alleviating the overwhelming demand for whole organs without exposing the recipient to the morbidity and mortality associated with a full organ transplantation.

Typically, stem cells are noted for their capacity for self-renewal and their ability to give rise to multiple differentiated cellular populations (Wagers et al., (2002) Gene Ther. 9:606-612). These characteristics can be referred to as stem cell capabilities. Stem cells can have a variety of different properties and categories of these properties. For example, in some forms stem cells are capable of proliferating for at least 10, 15, 20, 30, or more passages in an undifferentiated state. In some forms the stem cells can proliferate for more than a year without differentiating. Stem cells can also maintain a normal diploid karyotype while proliferating and/or differentiating. Some stem cells can maintain this normal karyotype through prolonged culture. Pluripotential stem cells, adult stem cells, blastocyst-derived stem cells, gonadal ridge-derived stem cells, teratoma-derived stem cells, totipotent stem cells, multipotent stem cells, embryonic stem cells (ES), embryonic germ cells (EG), and embryonic carcinoma cells (EC) are all examples of stem cells.

Stem cells are currently characterized by the presence and absence of specific lineage-related markers (Walkup et al., (2006) Stem Cells 24:1833-1840). Specifically, hepatic stem cells have been broadly characterized as a wide range of cell populations. Human livers contain two pluripotent cell types, hepatic stem cells and hepatoblasts, which both have distinct size, morphology, and gene expression profiles from that of mature hepatocytes (see Table 1). Each type of hepatic cell has unique phenotypic features. Hepatic stem cells, the precursors to hepatoblasts, have phenotypic features consisting of expression of Cytokeratin 19 (CK19), neuronal cell adhesion molecule (NCAM), epithelial cell adhesion molecule (EpCAM), and claudin-3; expression of low levels of albumin; and expression of alpha-fetoprotein and adult liver-specific proteins, (Schmelzer et al. (2006) Stem Cells 24:1852-1858). In contrast, hepatoblasts express high levels of albumin and alpha-fetoprotein (AFP), express low levels of adult liver-specific proteins and CK19, and do not express NCAM or CLDN-3. Mature hepatocytes, which exhibit a limited number of population doublings, express high levels adult liver-specific proteins, e.g., ASGP-R, and albumin, and lack expression of CK19, NCAM, EpCAM, CLDN-3, and AFP.

TABLE 1
Summary of Gene Expression

| | Hepatic | Hepato- | Mature |
|---|---|---|---|
| Gene | Stem Cells* | blasts* | Hepatocytes* |
| Cytokeratin 19 (CK 19) | High | Low | Absence |
| Neuronal cell adhesion | High | Absence | Absence |
| molecule (N-CAM) | | | |
| Epithelial cell adhesion | High | — | Absence |
| molecule (EpCAM) | | | |
| Claudin-3 (CLDN-3) | High | Absence | Absence |
| Albumin | Low | High | High |
| alpha-fetoprotein (AFP) | Absence | High | Absence |
| Adult liver specific proteins | Absence | Low | |

*Schmelzer et al., 2006. The phenotypes of pluripotent human hepatic progenitors. Stem Cells 24: 1852-1858

It is understood that hepatic progenitor cells can have any combination of any phenotypic features specific to human hepatic progenitor cells. For example, some stem cells can express CK19. In another example, some stem cells express a high level of CK 19. Another set of hepatic progenitor cells express a low level of Albumin. One set of hepatic progenitor cells expresses c-kit. Another set of hepatic stem cells, for example, can express CK19 and c-kit. Another set of hepatic progenitor cells, for example, could express CK19 and c-kit and a low level of Albumin.

In order to characterize the phenotypic features of the immortalized cell, mRNA expression can be compared to freshly isolated human hepatocytes by RT-PCR. For example, the expression of Cytokeratin 19, Neuronal cell adhesion molecule (NCAM), epithelial cell adhesion molecule (EpCAM), Claudin-3, albumin, alphafetoprotein, α1-antitrypsin, c-kit, adult liver-specific proteins, e.g., ASGP-R, and/or CYP 3A4 may be analyzed. In addition, protein expression of, for example, Cytokeratin 19, Neuronal cell adhesion molecule (NCAM), epithelial cell adhesion molecule (EpCAM), Claudin-3, albumin, alphafetoprotein, α1-antitrypsin, c-kit, adult liver-specific proteins, e.g., ASGP-R, and/or CYP 3A4, can be measured by Western immunoblotting. Karyotype analysis can be conducted by G-banding in a cytogenetic laboratory.

### Uses of Hepatic Progenitor Cells

### Cell Transplantation

Telomerase-immortalized human cells can be used to treat symptoms associated with the failure of differentiated organs and tissues that are amenable to organ or tissue transplantation. The immortalized cells can be used for transplantation into a patient in need thereof or, as appropriate, can be used as part of an extracorporeal organ support and direct cell transplantation treatments. In one embodiment, the puripotent immortalized cells of the instant invention may be used to treat any disorder that could be treated with stem cells, including malignancies (e.g., Acute lymphocytic leukemia (ALL), acute Myelogenouse Leukemia (AML), Chronic myelocytic leukemia (CML), and Myleodysplastic syndrome (MDS)), solid tumors (e.g., Liposarcoma, Neuroblastoma, Non-Hodgkin's lymphoma, Yolk sac sarcoma), hemoglobinopathies and various blood disorders (e.g., Amegakaryocytic thrombocytopenia (AMT), Aplastic anemia, Blackfan-Diamond anemia, Congenital cytopenia, Fanconi's anemia (genetic), Kostmann's syndrome (genetic), Sickle cell anemia, and Thalassemia), genetic metabolism disorders (e.g., Adrenoleukodystrophy, Bare-lymphocyte syndrome, Dyskeratosis congenita, Familial erythrophagocytic lymphohistiocytosis, Gaucher disease, Gunter disease, Hunter syndrome, Hurler syndrome (genetic), Inherited neuronal ceroid lipofuscinosis, Krabbe disease, Langerhans'-cell histiocytosis, Lesch-Nyhan disease, Leukocyte adhesion deficiency, and Osteopetrosis (genetic)), and immunodeficiencies (e.g., Adenosine deaminase deficiency (ADA or SCID-ADA), Severe combined immunodeficiency (SCIDs), Wiskott-Aldrich syndrome, and X-linked lymphoproliferative disease (XLP)). In a specific embodiment, conditions for which the telomerase-immortalized cells can be used include liver failure. Of particular interest is the treatment of symptoms of acute or chronic liver failure, due to for example fulminant hepatic failure (FHF), decompensated cirrhosis, drug overdose or other corporeal poisoning, or hepatic failure due to disease, such as hepatitis or cancer.

At present, there is no means to support a patient who has entered into end stage liver disease. Because the liver has the ability to regenerate, support for this short, crucial period can allow the patient to survive, either until a suitable organ is available or, in the best of circumstances, their own liver regenerates.

Several studies have observed the potential of progenitor cells in liver transplantation. One study transplanted fetal liver epithelial progenitor cells into syngeneic dipeptidyl peptidase IV mutant mice that had been subjected to liver injury (Sandhu et al., (2001) Am. J. Pathol. 159:1323-1334). These fetal liver epithelial progenitor cells continued to proliferate in the mice 6 months after transplantation, as opposed to a control group of transplanted mature hepatocytes. The immortalized neonatal human hepatocytes of the instant invention could be used for cellular transplantation in a similar manner.

In carrying out cellular transplantation, a sufficient number of immortalized cells to enable functional repopulation of a compromised organ or tissue are injected directly into the individual requiring treatment. In the application of telomerase-immortalized hepatocytes to direct hepatocyte transplantation, immortalized hepatocytes, generally in the amount of about 10% of a normal liver mass are injected intravenously (i.v.), intraperitoneally (i.p.), intrasplenically (i.s.), or directly intrahepatically (i.h.) into the patient in need thereof. Where the number of cells in a normal adult liver are estimated to be about 2.5×10¹¹ to 3.5×10¹¹ total cells, up to about 2.5×10¹⁰ to 3.5×10¹⁰ telomerase immortalized hepatocytes are injected in a cellular transplantation procedure. Depending on the size of the liver, the individual and the condition being treated, a lesser or greater number of cells are injected. The cells are administered in at least one treatment, but can be administered over several treatments. A maximum number of cells (i.e., about 10%) or a fraction of the maximum number of cells (up to 10%) are administered in each of one or more treatments. Generally, one treatment is sufficient for the immortalized hepatocytes to proliferate and appropriately associate themselves with the endogenous liver tissue, such that normal liver function is regenerated, even if the endogenous liver tissue does not itself regenerate. Additional treatments are administered if necessary.

Following treatment, the patient is evaluated to determine whether symptoms have been alleviated. Both the biological efficacy of the treatment modality as well as the clinical efficacy are evaluated, if possible. The clinical efficacy, i.e., whether treatment of the underlying effect is effective in changing the course of disease, can be more difficult to measure. While the evaluation of the biological efficacy goes a long way as a surrogate endpoint for the clinical efficacy, it is not definitive. Thus, measuring a clinical endpoint which can give an indication of the presence of functioning immortalized cells after, for example, a six-month period of time, can give an indication of the clinical efficacy of the treatment.

An example of a device created to support a patient in end stage liver disease has been developed and tested in animals and on several patients in the United States and Great Britain (Sussman et al., (1992) Hepatology 16, 60-65; Sussman et al., (1994) Artificial Organs 18, 390-396; Millis et al., (2002) Transplantation 74, 1735-1746). In this device, a hollow fiber cartridge is filled with a human liver cell line. The cells are separated from the patient's immune system by the cellulose acetate fibers. Blood is pumped through the lumen of the fibers, and small molecules diffuse through the fibers to the cells, where they are appropriately metabolized. Evidence suggests that the device, although crude, is fairly effective. Other similar devices, using animal hepatocytes, also appear to be effective (Hui et al., (2001) J. Hepatobiliary Pancreat Surg. 8, 1-15).

The problem arises in that there is no source of hepatocytes to fill the device. In order to be effective, each device requires about 200 g of cells, 15 to 20% of the total liver mass. Hepatocytes, despite their regenerative capabilities in vivo, do not divide to any extent in culture. This problem has been approached by employing a tumor-derived human liver cell line, which is immortalized (Sussman et al., (1995) Scientific American: Science and Medicine 2, 68-77). These cells supply a constantly renewable, reproducible and unlimited supply of devices.

Unfortunately, the tumor-derived source of these cells has presented acceptance and regulatory problems for its use in human therapy. The disclosed immortalized neonatal human hepatocytes produced from the compositions and methods disclosed herein can circumvent these hurdles.

### Toxicology Screening

The desire of the pharmaceutical industry to drive down the staggering cost of new drug discovery and development has forced an examination of the factors that cause drug candidates to fail. After efficacy problems, the most common reason for failure is toxicity (van de Waterbeemd et al., (2003) Nat. Rev. Drug Disc. 2, 192-204). Troglitazone and trovafloxacin are well known examples of compounds which were pulled or whose use was severely curtailed due to liver toxicity (Suchard (2001) Int. J. Med. Toxicol. 4, 15-20).

Ideally, the toxic properties of new compounds can be recognized and avoided early in drug development. Compounds can be screened through a battery of tests at multiple concentrations to develop a structural ranking that can be used by the chemists to direct the next round of synthesis. In this way, the toxic properties of a compound can be minimized while maximizing the therapeutic properties.

The development of an immortalized neonatal human hepatocyte cell line that exhibits the features of a hepatic progenitor cell will allow the testing of compound toxicity in vitro, raising the probability of success in clinical trials. By testing the compounds in the toxicology assays, a clear picture of the toxic potential of new compounds can be determined before testing in humans. This will have a dramatic effect on the cost and speed of new drug development since clinical testing is by far the most expensive phase.

### Pharmacokinetics

The desire of the pharmaceutical industry to drive down the staggering cost of new drug discovery and development has forced an examination of the factors that cause drug candidates to fail. The development of an immortalized neonatal human hepatocyte cell line that exhibits the features of a hepatic progenitor cell will allow the testing of compound pharmacokinetics in vitro, raising the probability of success in clinical trials. Pharmacokinetic parameters predictable by the present invention include those employed in the ordinary course of drug development. Without limitation, these include Cₘₐₓ and C_{depot}. The common understanding of these terms by the artisan is applicable herein. By way of example only in this regard: Cₘₐₓ is typically the maximum concentration of drug measured in serum (e.g., blood) after administration. The time it takes to reach Cₘₐₓ is denoted tₘₐₓ; for example, in an embodiment of the invention Cₘₐₓ for various formulations can be generally manifested in about 15 minutes to about 30 minutes. C_{depot} (depot level) is typically the average serum concentration between set time periods, e.g., the average concentration measured periodically between 12 hrs and 14 days.

In practice, the concentration of the drug compound in an in vitro assay is determined by means known in the art. Concentrations in this regard may be measured at one or more points in time, e.g., after 15 min, 1 hr, 24 hrs or up to about 7 days or more, e.g., 14 days. Concentration thus determined according to the present invention is correlated with various in vivo parameters aforesaid such as Cₘₐₓ and/ C_{depot}.

Correlations serviceable for the invention can be obtained by any manner known to the art. By way of example only, correlations can be obtained by pre-establishing profiles for the pharmacokinetic parameters of concern (e.g., Cₘₐₓ, depot level) in suitable models using one or more formulations comprising the poorly soluble drug compound of interest. The pre-established profiles can then be statistically assessed against the concentrations of the same formulations as measured in the supernatant of the immortalized neonatal human hepatocytes as aforesaid. Any statistical method can be utilized to compare the two data sets that result (pre-established and supernatant), e.g., linear regression analysis. In vivo performance of other formulations comprising the poorly soluble drug compound can thereafter be predicted by correlating the supernatant concentrations of same to the parameters determined as aforesaid.

### Drug Metabolism

Immortalized neonatal human hepatocytes according to the present invention can be used in a method for evaluating the metabolism of a compound by human liver. Currently, the metabolism, toxicity, and carcinogenicity of chemical compounds, is typically examined using laboratory animal such as rat, dog or hog. However, since differences between the metabolic pathway of human and laboratory animals is obvious, circumspection is required in order to apply the data of laboratory animals to human. The immortalized cell according to the present invention exhibits phenotypic features of a hepatic progenitor cell and has great significance as a new in vitro assay model for drug metabolism. Concretely, it may be used for analyzing the metabolism of a drug in liver cells, studying the interaction of drugs, and assaying for the production of a mutagenic substance derived from a drug in the liver.

### Gene Therapy

Immortalized neonatal human hepatocyte cell lines may also be used in gene therapy. Generally, the preparation of immortalized neonatal human hepatocyte cells of the invention may be used to deliver a therapeutic gene to a patient that has a condition that is amenable to treatment by the gene product of the therapeutic gene. In one embodiment, the pluripotent immortalized cells of the instant invention may be used to treat any stem cell disorder, including malignancies (e.g., Acute lymphocytic leukemia (ALL), acute Myelogenouse Leukemia (AML), Chronic myelocytic leukemia (CML), and Myleodysplastic syndrome (MDS)), solid tumors (e.g., Liposarcoma, Neuroblastoma, Non-Hodgkin's lymphoma, Yolk sac sarcoma), hemoglobinopathies and various blood disorders (e.g., Amegakaryocytic thrombocytopenia (AMT), Aplastic anemia, Blackfan-Diamond anemia, Congenital cytopenia, Fanconi's anemia (genetic), Kostmann's syndrome (genetic), Sickle cell anemia, and Thalassemia), genetic metabolism disorders (e.g., Adrenoleukodystrophy, Bare-lymphocyte syndrome, Dyskeratosis congenita, Familial erythrophagocytic lymphohistiocytosis, Gaucher disease, Gunter disease, Hunter syndrome, Hurler syndrome (genetic), Inherited neuronal ceroid lipofuscinosis, Krabbe disease, Langerhans'-cell histiocytosis, Lesch-Nyhan disease, Leukocyte adhesion deficiency, and Osteopetrosis (genetic)), and immunodeficiencies (e.g., Adenosine deaminase deficiency (ADA or SCID-ADA), Severe combined immunodeficiency (SCIDs), Wiskott-Aldrich syndrome, and X-linked lymphoproliferative disease (XLP)). In a specific embodiment, the immortalized neonatal hepatocytes are particularly useful to deliver therapeutic genes that are involved in or influence liver disease (e.g., α-1-antitrypsin to treat Alpha-1 Antitrypsin Deficiency). Methods for gene therapy are known in the art. See for example, U.S. Pat. No. 5,399,346 by Anderson et al. A biocompatible capsule for delivering genetic material is described in PCT Publication WO 95/05452 by Baetge et al. Methods of gene transfer into bone-marrow derived cells have also previously been reported (see U.S. Pat. No. 6,410,015 by Gordon et al.). The therapeutic gene can be any gene having clinical usefulness, such as a gene encoding a gene product or protein that is involved in disease prevention or treatment, or a gene having a cell regulatory effect that is involved in disease prevention or treatment. The gene products should substitute a defective or missing gene product, protein, or cell regulatory effect in the patient, thereby enabling prevention or treatment of a disease or condition in the patient.

Accordingly, the invention further provides a method of delivering a therapeutic gene to a patient having a condition amenable to gene therapy comprising: (i) selecting the patient in need thereof, (ii) modifying the preparation of claim 1 so that the cells of the preparation carry a therapeutic gene; and (iii) administering the modified preparation to the patient. The preparation may be modified by techniques that are generally known in the art. The modification may involve inserting a DNA or RNA segment encoding a gene product into the mammalian immortalized neonatal hepatic cells, where the gene enhances the therapeutic effects of the immortalized neonatal hepatic cells. The genes are inserted in such a manner that the modified immortalized neonatal hepatic cell will produce the therapeutic gene product or have the desired therapeutic effect in the patient's body. The gene can be inserted into the immortalized neonatal hepatic cells using any gene transfer procedure, for example, direct injection of DNA, receptor-mediated DNA uptake, retroviral-mediated transfection, viral-mediated transfection, non-viral transfection, lipid based transfection, electroporation, calcium phosphate mediated transfection, microinjection or proteoliposomes, all of which may involve the use of gene therapy vectors. Other vectors can be used besides retroviral vectors, including those derived from DNA viruses and other RNA viruses. As should be apparent when using an RNA virus, such virus includes RNA that encodes the desired agent so that the immortalized neonatal hepatic cells that are transfected with such RNA virus are therefore provided with DNA encoding a therapeutic gene product.

In accordance with another aspect of the invention, a purified preparation of mammalian immortalized neonatal hepatic cells, in which the cells have been modified to carry a therapeutic gene, may be provided in containers or commercial packages that further comprise instructions for use of the preparation in gene therapy to prevent and/or treat a disease by delivery of the therapeutic gene. Accordingly, the invention further provides a commercial package comprising a preparation of mammalian immortalized neonatal hepatic cells of the invention, wherein the preparation has been modified so that the cells of the preparation carry a therapeutic gene, and instructions for treating a patient having a condition amenable to treatment with gene therapy.

### Production of Fully Differentiated Hepatocytes

The immortalized cell or population of cells of the present invention can be induced to differentiate to form a number of cell lineages, including, for example, fully differentiated hepatocytes. In order to produce differentiated hepatocytes, immortalized neonatal human hepatocyte cell lines can be incubated with any known differentiation medium, e.g., a differentiation medium containing hepatocyte growth factor (HGF), fibroblast growth factor-4 (FGF-4), appropriate growth factors, chemokines, cytokines, or LIF (leukemia-inhibiting factor).

Differentiated, or mature, hepatocytes have size, morphology, and gene expression profiles that are distinct from those of immortalized neonatal human hepatocytes (see Table 1). In order to characterize the phenotypic features of the differentiated hepatocytes, mRNA expression can be compared to freshly isolated human hepatocytes by RT-PCR. For example, the expression of Cytokeratin 19, Neuronal cell adhesion molecule (NCAM), epithelial cell adhesion molecule (EpCAM), Claudin-3, albumin, alphafetoprotein, α1-antitrypsin, c-kit, adult liver-specific proteins, e.g., ASGP-R, and/or CYP 3A4 may be analyzed. In addition, protein expression of, for example, Cytokeratin 19, Neuronal cell adhesion molecule (NCAM), epithelial cell adhesion molecule (EpCAM), Claudin-3, albumin, alphafetoprotein, α1-antitrypsin, c-kit, adult liver-specific proteins, e.g., ASGP-R, and/or CYP 3A4, can be measured by Western immunoblotting. Karyotype analysis can be conducted by G-banding in a cytogenetic laboratory.

The invention provides numerous uses for the differentiated hepatocyte cells. For example, fully differentiated hepatocytes can be used in cell transplantation, toxicology screening, pharmokinetics, drug metabolism, and gene therapy (as described above). In addition, they can be used for studying cell senescence.

### EXAMPLES

### Introduction to the Examples

Primary hepatocytes have been extensively used in a wide variety of experimental studies, however, limited lifespan as well as restricted availability are major constraints for such studies. Immortalization of primary cells extends their replicative capacity and would provide for continuous, unlimited availability. Immortalized hepatocytes with a stable phenotype that mimics the original tissue would constitute very attractive experimental models for use in toxicological and pharmaceutical studies. Ectopic expression of human telomerase reverse transcriptase (hTERT) is one of the major strategies used in developing immortalized cells and allows for the retention of the original cellular characteristics to a large extent and avoids some of the problems associated with other approaches. The cell lines NeHepLxHT, NeHepMsHT, and NeHepBaHT, were developed from neonatal human hepatocytes by transduction with retroviral expression vectors containing the hTERT gene. The cell lines were continuously cultured for more than twenty five passages without senescence whereas the parental cells senesced within three to five passages. Thus, induction of stable expression of hTERT in the neonatal cells led to immortalization of these cells. Analysis of telomerase activity, by telomeric repeat amplification protocol (TRAP) assay, indicated elevated levels of telomerase activity in these cells compared to the parental cells. The immortalized cell line maintained a diploid karyotype and expressed gene product profiles similar to normal neonatal human hepatocytes. These data suggest that this immortalized cell line preserved the normal biological characteristics of neonatal hepatocytes and may therefore be useful models for in vitro studies.

Neonatal human hepatocytes were immortalized using the retroviral gene transfer and expression systems as described above. Briefly, neonatal hepatocytes were transfected with one of three retroviral viruses containing the hTERT gene. The cell line transfected with pBABE puro containing the hTERT gene was named NeHepBaHT. The cell line transfected with pLXSN containing the hTERT gene was named NeHepLxHT, and the cell line transfected with pMSCV containing the hTERT gene was named NeHepMsHT. Cells were cultured in medium containing either G418 or puromycin until stable clones were selected. These hTERT-immortalized neonatal human hepatocytes were free of microbial contamination, had a stable genotype and phenotype, had an extended lifespan, and expressed the telomerase protein.

### Example 1

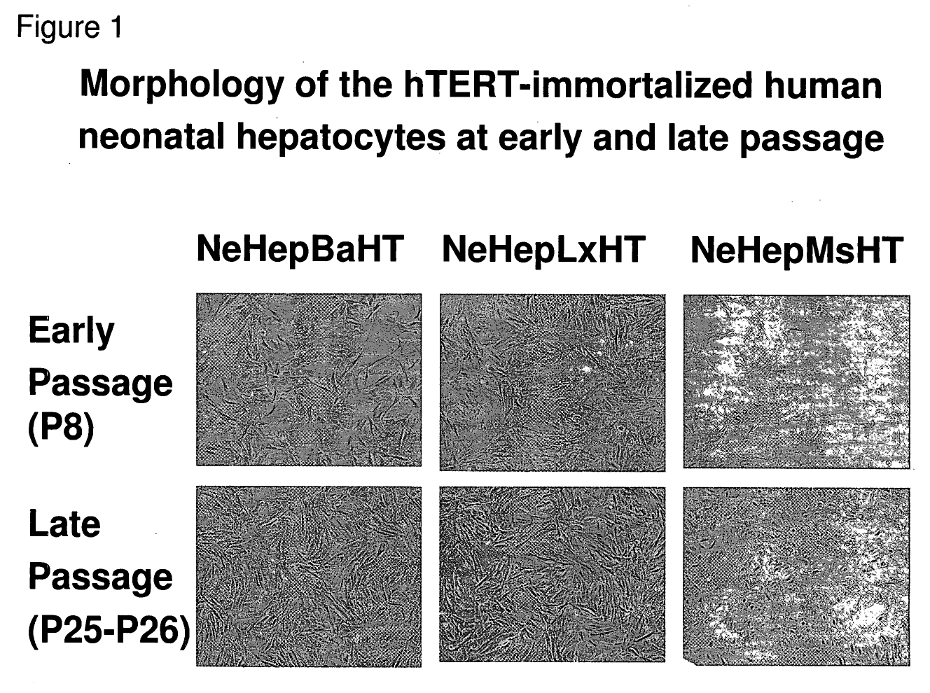

The morphology of the hTERT-immortalized neonatal human hepatocytes was observed at early passage (passage 8) and late passage (passage 25-26) (FIG. 1). As can be observed in FIG. 1, the morphology of the immortalized cell lines NeHepLxHT, NeHepMsHT, and NeHepBaHT is similar to that of hepatocytes, both at early and late passage. Furthermore, all three cell lines show a consistent morphology between early and late passage, suggesting that the phenotypic characteristics of the immortalized cell are maintained in late passage.

### Example 2

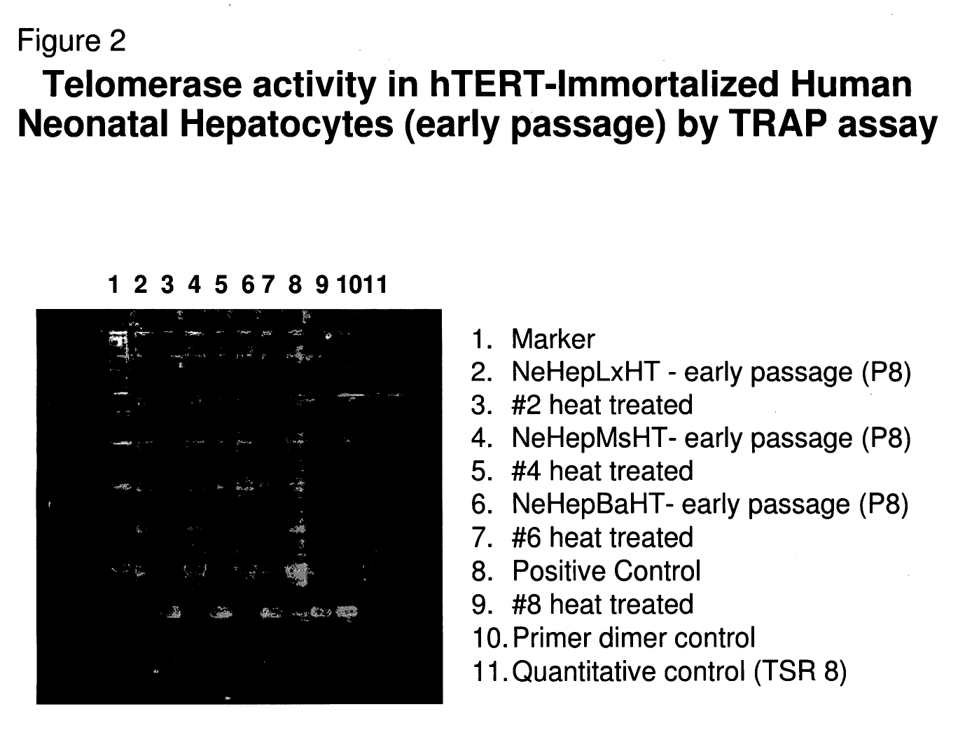

The TRAP assay was used to determine telomerase activity of the hTERT-immortalized neonatal human hepatocytes at early passage (FIG. 2). Lane 2 contains the NeHepLxHT immortalized cells and indicates that telomerase is expressed in this cell line at early passage. Similarly, lanes 4 and 6 indicate that the immortalized cells NeHepMsHT and NeHepBaHT, respectively, express telomerase similar to the quantitative control at early passage (lane 11). Thus, the immortalized cell lines all express telomerase.

### Example 3

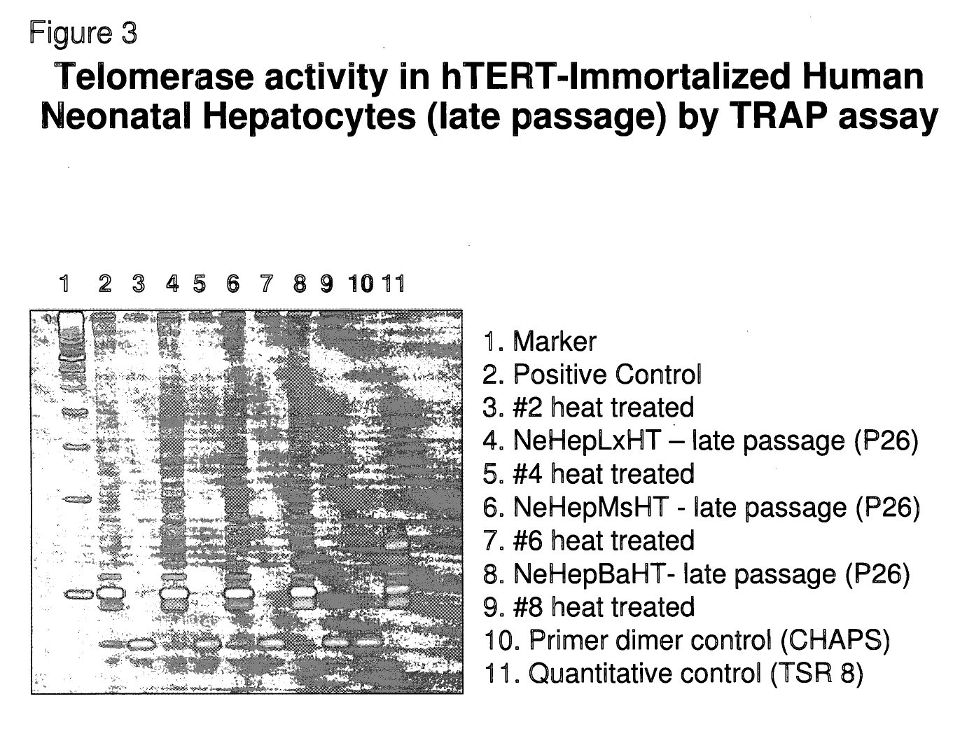

The TRAP assay was used to determine telomerase activity of the hTERT-immortalized neonatal human hepatocytes at late passage (FIG. 3). Lane 2 contains the NeHepLxHT immortalized cells and indicates that telomerase is expressed in this cell line at late passage. Similarly, lanes 4 and 6 indicate that the immortalized cells NeHepMSHT and NeHepBaHT, respectively, express telomerase similar to the quantitative control at late passage (lane 11).

### Example 4

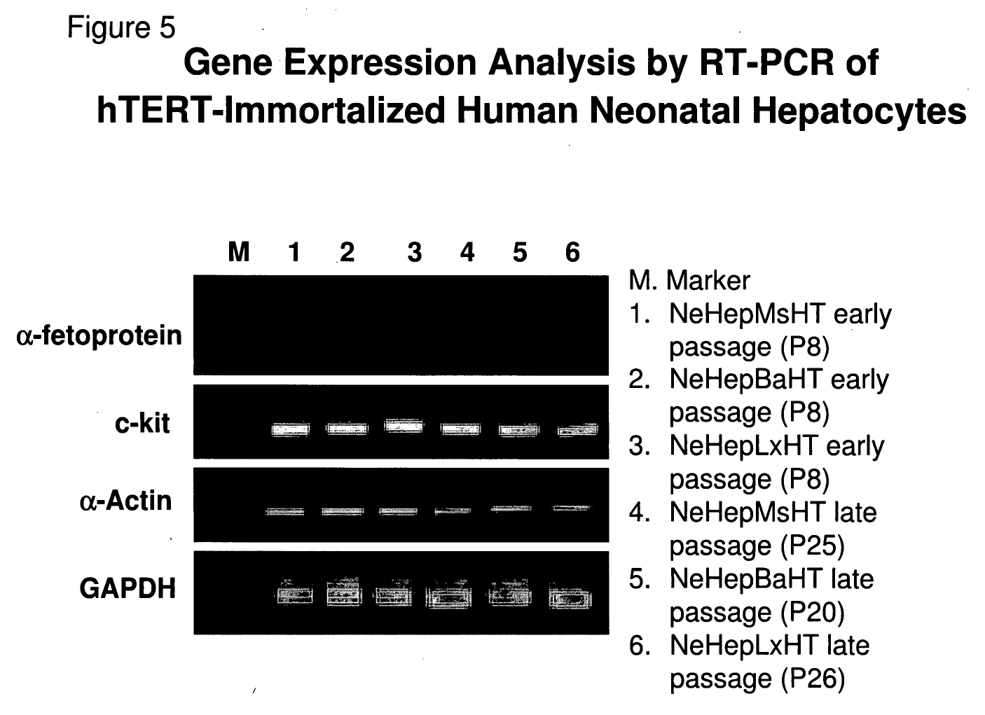

Analysis of gene expression by RT-PCR was performed using the hTERT-immortalized neonatal human hepatocytes at early and late passage (FIGS. 4 and 5). A summary of these results can also be found in Table 2. In all immortalized cell lines, at both early and late passage, expression of the housekeeping genes α-actin, β-actin, and GAPDH was found to be positive (FIGS. 4 and 5). Expression of the hepatospecific gene, α1-antitrypsin, was also found to be positive in the immortalized neonatal human hepatic cell lines at all stages of passage (FIG. 4).

Expression of the adult liver-specific protein, Asialoglycoprotein receptor (ASGP-R) (FIG. 4), and expression of α-fetoprotein (AFP) (FIG. 5), however, were absent in the immortalized neonatal human hepatic cell lines at all stages of passage (FIG. 5). On the other hand, c-kit (FIG. 5), a cell surface marker associated with hematopoietic stem cells, was expressed in the immortalized cell lines. The expression of albumin was found to be positive but low in comparison to the housekeeping gene, β-actin.

The expression of CK19 in the immortalized neonatal human hepatocytes (Table 2) was found to be high as compared to the expression of a housekeeping gene in all cell lines at early passage. In the cell line NeHepBaHT, CK19 expression was reduced as compared to the expression of a housekeeping gene in late passage, while in the cell line NeHepLxHT, CK19 expression was absent in late passage. NeHepMsHT, however, maintained a high level of CK 19 expression in late passage as compared to the expression of a housekeeping gene.

TABLE 2
Summary of Gene Expression

| | Cell Type | | | | | | | | |
|---|---|---|---|---|---|---|---|---|---|
| | Hepatic | | Mature | NeHepBaHT | | NeHepLxHT | | NeHepMsHT | |
| Gene | Stem Cells * | Hepatoblasts * | Hepatocytes * | Early | Late | Early | Late | Early | Late |
| Cytokeratin 19 | High | Low | Absence | Positive | Reduced | Positive | Negative | Positive | Positive |
| (CK 19) | | | | | | | | | |
| Neuronal cell | High | Absence | Absence | Positive | Positive | Positive | Positive | Positive | Positive |
| adhesion | | | | | | | | | |
| molecule (N- | | | | | | | | | |
| CAM) | | | | | | | | | |
| Epithelial cell | High | — | Absence | Positive | Positive | Positive | Positive | Positive | Positive |
| adhesion | | | | | | | | | |
| molecule | | | | | | | | | |
| (EpCAM) | | | | | | | | | |
| Claudin-3 | High | Absence | Absence | Positive | Positive | Positive | Positive | Positive | Positive |
| (CLDN-3) | | | | | | | | | |
| Albumin | Low | High | High | Positive/ | Positive/ | Positive/ | Positive/ | Positive/ | Positive/ |
| | | | | Low | Low | Low | Low | Low | Low |
| alpha- | Absence | High | Absence | Negative | Negative | Negative | Negative | Negative | Negative |
| fetoprotein | | | | | | | | | |
| (AFP) | | | | | | | | | |
| Adult liver | Absence | Low | — | Negative | Negative | Negative | Negative | Negative | Negative |
| specific proteins | | | | | | | | | |
| e.g., ASGP-R | | | | | | | | | |
| CYP 3A4 | | | | Negative | Negative | Negative | Negative | Negative | Negative |
| α1-antitrypsin | | | | Positive | Positive | Positive | Positive | Positive | Positive |
| c-kit | | | | Positive | Positive | Positive | Positive | Positive | Positive |

* Schmelzer et al., 2006. The phenotypes of pluripotent human hepatic progenitors. Stem Cells 24: 1852-1858

### REFERENCES

Amicone et al., (1997) EMBO J. 16 (3):495-503
Cech et al., U.S. Pat. No. 6,166,178
Danos and Mulligan. 1988. Proc. Natl. Acad. Sci. (USA) 85: 6460-6464
Dimri et al., (1995) Proc Natl Acad Sci USA 92: 9363-9367
Finer et al., U.S. Pat. No. 5,686,279
Greider et al., (1985) Cell 43:405-413
Hayflick et al., (1961) Exp. Cell Res. 25:585-621
Hui et al., (2001) J. Hepatobiliary Pancreat Surg. 8:1-15
Kilian, et al. (1997) Hum Mol Genet. 6: 2011
Kitamura et al., (1995) Proc. Natl. Acad. Sci. 92: 9146-9150
Kobayashi et al., (2000) Science 287:1258-62
Lieber et al., (1975) Proc. Natl. Acad. Sci. USA 72:2315-2319
Markowitz, et al. (1988) J. Virol. 62: 1120-1124
Marowitz et al., (1988) Virology 167: 400-406
Mayerson, et al. (1997) Cell 90: 78
Miller and Buttimore, (1986) Mol. Cell. Biol. 6: 2895-2902
Miller and Rosman, (1989) BioTechniques 7: 980-990
Miller, (1990), Hum. Gene Ther. 1-5
Miller and Miller, (1994) J. Virol. 68: 8270-8276
Miller, (1996) Proc. Natl. Acad. Sci. (USA) 93: 11407-11413
Millis et al., (2002) Transplantation 74, 1735-1746
Morgenstern and Land, (1990)
Nakamura, et al. (1997) Science 277: 955
Nakamura et al., (1997) Transplantation 63 (11):1541-47
Pear et al., (1993) Proc. Natl. Acad. Sci. 90: 8392-8396
Ray et al., (2000) Virology 271:197-204
Sambrook and Russell, (2001) Molecular Cloning, 3rd Edition
Sandhu et al., (2001) Am. J. Pathol. 159:1323-1334
Schmelzer et al., 2006. Stem Cells 24:1852-1858
Strom, et al. (1996) Methods Enzymol 272:388-401
Suchard (2001) Int. J. Med. Toxicol. 4, 15-20
Sussman et al., (1992) Hepatology 16, 60-65
Sussman et al., (1994) Artificial Organs 18, 390-396
Sussman et al., (1995) Scientific American: Science and Medicine 2, 68-77
Temin, (1990) Hum. Gene Ther. 1-111
Wagers et al., (2002) Gene Ther. 9:606-612
Walkup et al., (2006) Stem Cells 24:1833-1840
Weiss et al., (1984) RNA Tumor Viruses, New York
Werner et al., (2000) Biotechnol Bioeng 68 (1): 59-70
van de Waterbeemd et al., (2003) Nat. Rev. Drug Disc. 2, 192-204
Vaziri and Benchimol (1998) Curr Biol 8: 279-282

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A population of immortalized human cells that express a human telomerase, wherein the cells exhibit phenotypic features of human hepatic progenitor cells at early passage in vitro and continues to express said phenotypic features at late passage in vitro.

2. An immortalized human cell that expresses a human telomerase, wherein the cell exhibits phenotypic features of human hepatic progenitor cells at early passage in vitro and continues to express said phenotypic features at late passage in vitro.

3. A differentiated hepatocyte produced from the immortalized cell or population of cells of claim 1 or 2.

4. The immortalized cell or cells of claim 1 or 2, wherein the cell or population of cells express c-kit.

5. The immortalized cell or cells of claim 4, wherein Cytokeratin 19 is expressed at a low level.

6. The immortalized cell or cells of claim 1 or 2, wherein the cell or population of cells express Albumin at a low level.

7. The immortalized cell or cells of claim 1 or 2, wherein the cell or population of cells express c-kit.

8. The immortalized cell or cells of claim 1 or 2, wherein the telomerase is encoded by a low level.

9. The immortalized cell or cells of claim 1 or 2, wherein the cell or cells are diploid.

10. The immortalized cell or cells of claim 9, wherein the cell or cells are characterized by one or more of: expression system containing the hTERT gene.

11. The immortalized cell or cells of any one claims 1 or 2, wherein the cell or cells have a retroviral gene transfer and expression system.

12. The immortalized cell or cells of any one of claims 1-2, wherein the cell is pLXSN.

13. The immortalized cell or cells of claim 11, wherein the retroviral gene transfer and expression system is pMSCV.

14. The immortalized cell or cells of claim 13, wherein the retroviral gene transfer and expression of Cytokeratin 19.

15. The immortalized cell or cells of claim 13, wherein the retroviral gene transfer and expression system is pLXSN.

16. The immortalized cell or cells of claim 13, wherein the retroviral gene transfer and expression system is pMSCV.

17. The immortalized cell or cells of claim 16, wherein the cell or cells exhibit phenotypic features in late passage in vitro.

18. The immortalized cell or cells of claim 17, wherein Cytokeratin 19 is expressed at a high level.

19. The immortalized cell or cells of claim 17, wherein the cell or cells express Albumin at a low level.

20. The immortalized cell or cells of claim 17, wherein the cell or cells express c-kit.

21. The immortalized cell or cells of claim 17, wherein Cytokeratin 19 is expressed at a low level.

22. The immortalized cell or cells of claim 17, wherein the cell or cells express Albumin at a low level.

23. The immortalized cell or cells of claim 17, wherein the cell cells express c-kit.

24. The immortalized cell or cells of claim 17, wherein the cell or cells express Cytokeratin 19 and expresses Albumin at a low level.

25. The immortalized cell or cells of claim 17, wherein the cell or cells express Cytokeratin 19, expresses Albumin at a low level and expresses c-kit.

26. The immortalized cell or of claim 17, wherein the phenotype of the cell or population of cells is characterized by one or more of: expression of Cytokeratin 19, expression of NCAM, expression of EpCAM, expression of CLDN-3; low expression of Albumin; the absence of expression of alpha-fetoprotein, the absence of expression of ASGP-R, and the absence of expression of CYP 3A4.

27. A method of obtaining an immortalized human hepatocyte having the phenotypic features of human hepatic progenitor cells, said method comprising the steps of:
(i) introducing an exogenous nucleic acid molecule encoding a human telomerase into a neonatal human hepatocyte to obtain a transfected neonatal human hepatocyte cell that expresses the exogenous human telomerase; and
(ii) propagating the transfected human hepatocyte cell in vitro,
to thereby obtain an immortalized neonatal human hepatocyte cell that exhibits phenotypic features of human hepatic progenitor cells at early passage in vitro and continues to express said phenotypic features at late passage in vitro.

28. The method of claim 27, wherein the nucleic acid molecule comprises a retroviral gene transfer and expression system and a telomerase.

29. The method of claim 27, wherein the immortalized neonatal human TERT gene.

30. The method of claim 27, wherein the retroviral gene transfer and expression system is pBABE Puro.

31. The method of claim 27, wherein the retroviral gene transfer and expression system is pLXSN.

32. The method of claim 27, wherein the retroviral gene transfer and expression system is pMSCV.

33. The method of claim 27, wherein the immortalized neonatal human hepatocyte expresses c-kit.

34. The method of claim 33, wherein the immortalized neonatal human hepatocyte expresses Cytokeratin 19 at a low level.

35. The method of claim 27, wherein the immortalized neonatal human hepatocyte expresses Albumin at a low level.

36. The method of claim 27, wherein the immortalized neonatal hepatocyte expresses c-kit.

37. The method of claim 27, wherein the immortalized neonatal human hepatocyte expresses Cytokeratin 19 and expresses Albumin at a low level.

38. The method of claim 27, wherein the immortalized neonatal human hepatocyte expresses Cytokeratin 19, expresses Albumin at a low level, and expresses c-kit.

39. The method of claim 27, wherein the phenotype of the immortalized neonatal hepatocyte is characterized by one or more of: expression of Cytokeratin 19, expression of NCAM, expression of EpCAM, expression of CLDN-3; low expression of Albumin; the absence of expression of alpha-fetoprotein, the absence of expression of ASGP-R, and the absence of expression of CYP 3A4.

40. A method of ameliorating at least one symptom of disease in an individual in need thereof, said method comprising the step of: transplanting to said individual the immortalized cell of any one of claims 1 or 2, whereby at least one symptom of hepatic disease is ameliorated.

41. A method of evaluating the toxicity of a compound, said method comprising the steps of: contacting the immortalized cell of any one of claims 1 or 2 with the compound; measuring the toxicity of the compound for the immortalized cells; to thereby evaluate the toxicity of a compound.

42. A method of evaluating the pharmacokinetics of a compound in vitro, said method comprising the steps of: contacting the immortalized cell of any one of claims 1 or 2 with the compound; measuring the pharmacokinetics of the compound for the immortalized cells; to thereby evaluate the pharmacokinetics of a compound.

43. A method of evaluating the metabolism of a compound, said method comprising the steps of: contacting the immortalized cell of any one of claims 1 or 2 with the compound; measuring the metabolasis of the compound for the immortalized cells; to thereby evaluate the metabolism of a compound.

44. A method of delivering a therapeutic gene to a patient having a condition amenable to gene therapy comprising:
(i) selecting the patient in need thereof;
(ii) introducing a therapeutic gene into the immortalized cell of claims 1 or 2 to obtain a modified cell or population of cells; and
(iii) administering the modified cell or population of cells to the patient.

45. A commercial package comprising the immortalized cell or population of cells of claims 1 or 2, wherein a therapeutic gene has been introduced into the immortalized cell or population of cells to obtain a modified cell or population of cells, and instructions for treating a patient having a condition amendable to treatment with gene therapy.
